(19) United States
(12) Patent Application Publication
(10) Pub. No.: US 2020/0069296 A1
(43) Pub. Date: Mar. 5, 2020

(54) APPARATUS AND METHOD TO ISOLATE SPERM BASED ON PLANAR-CONFINED SWIMMING

(71) Applicants: FARHANG TARLAN, NORTH YORK (CA); JASON RIORDON, TORONTO (CA); DAVID SINTON, TORONTO (CA); THE GOVERNING COUNCIL OF THE UNIVERSITY OF TORONTO, Toronto (CA)

(72) Inventors: FARHANG TARLAN, NORTH YORK (CA); JASON RIORDON, TORONTO (CA); DAVID SINTON, TORONTO (CA)

(21) Appl. No.: 16/553,728

(22) Filed: Aug. 28, 2019

Related U.S. Application Data
(60) Provisional application No. 62/723,743, filed on Aug. 28, 2018.

(51) Int. Cl. A61B 10/00

(52) U.S. Cl. CPC A61B 10/0058; A61D 19/00; A61B 10/0096

(57) ABSTRACT

The present invention provides a microfluidic device with a confined geometry for isolating a slither-capable subpopulation of sperm which is of higher quality than the raw sperm population. The proposed device isolates slither-capable sperm based on their ability to enter and traverse a confined region where 3D locomotion is restricted. The DNA integrity of the selected sperm was higher than that of the corresponding raw samples by 55% and 122% for donors and patients, respectively. In side-by-side testing this method outperforms current clinical selection methods, density gradient centrifugation and swim-up, as well as sperm selected via general motility. Slithering represents a viable selection mechanism, readily applicable to clinical workflows with the potential to improve outcomes for couples and offspring.

### FIELD

The present disclosure relates to the isolation of sperm of high quality for fertility clinics and more particularly the present disclosure relates to a microfluidic device which can isolate a 2D slither-swimming capable subset of sperm from a semen sample.

### BACKGROUND

With the global decline in fertility rates and an increased understanding of male factors in assisted reproduction, selection of high quality sperm via slithering presents a clinically-applicable approach to improve fertility treatment outcomes. Providing a simple, yet functional solution to high-quality sperm selection is a major challenge in assisted reproduction.

In vitro, the majority of established clinical sperm selection methods rely on sperm motility in bulk fluid as their underlying mechanism of selection, the most notable of which is the swim-up assay. In swim-up, motile sperm swim into a culture medium, leaving behind weaker or damaged sperm. Researchers have improved on this motility-based strategy by incorporating biophysical cues.

Sperm locomotion is critical for natural fertilization in vivo as well as assisted reproduction in vitro. In vivo, human sperm traverse long and convoluted paths through highly viscous media in the female reproductive tract to reach the egg. Scanning electron micrographs of the female reproductive tract indicate that a large number of sperm traverse much of this distance in micrometer-sized longitudinal grooves.

Sperm have been shown to respond to chemical gradients, temperature gradients, fluid flow, or surface architecture. Some of these cues have been used as a basis for sperm selection in conjunction with sperm motility in bulk fluid. These sperm selection techniques isolate higher quality sperm from a raw human semen sample and are known to influence the success rate of the assisted reproduction cycle as well as the health and fertility potential of the offspring. All of these selection approaches are based on sperm swimming in environments that allow for a spiral tail beat pattern in three-dimensional (3D) space.

In practice, sperm tend to accumulate and navigate near surfaces. Near-surface navigation in vivo has been shown to help sperm swim against a pathogen-rejecting flow. Next to surfaces, sperm can exhibit a two-dimensional (2D) slithering mode, in a manner distinct from bulk or near-wall swimming. In the slither swimming mode, the entire length of the sperm lies within 1 μm of the substrate, and the sperm tail beats on a plane parallel to the surface. In the case of human sperm, the slither swimming mode has only been observed at high viscosities. Particularly at 40 mPa·s, over 65% of human sperm have been shown to exhibit slither mode swimming.

It has also been shown that human sperm swim 50% faster in the slither swimming mode than in the bulk mode. The faster speeds have been attributed to the reduced drag force and increased efficiency of this planar sperm locomotion. The high viscosity of the female reproductive tract indicates that slither swimming ability could play a role in facilitating natural conception. Collectively, previous studies established the preference of sperm to slither at surfaces in viscous media and indicated the biological utility of this swimming mode in vivo. The proposed microfluidic device functions to induce the slither swimming of sperm within a high viscosity environment in an attempt to select a sperm sample for fertilization with a higher DNA integrity.

### SUMMARY

The present disclosure provides a microfluidic device for isolating sperm of a desired quality, the device comprising:
a) an inlet reservoir which for holding a raw sperm sample;
b) an outlet reservoir for collecting sperm separated from the sample; and
c) one or more selection channels disposed between said inlet reservoir and said selection channels to provide fluid communication between the inlet reservoir and outlet reservoir, the one or more selection channels being geometrically configured to impede helical locomotion of a subpopulation of sperm and allow 2D slither swimming of a slither capable subset of sperm within the said one or more selection channels.

The one or more selection channels may have a dimension that is sufficiently low so as to restrict the helical locomotion of sperm within the one or more selection channels.

The one or more selection channels may have a channel dimension in a range from 1.0 μm to 10.0 μm.

A length of each of said one or more selection channels may be in a range from 1 μm to 10 cm and encourages a slither swimming subset of sperm to traverse the one or more selection channels.

The one or more selection channels may have a channel dimension which is a channel height of 2 μm, and wherein the length of each selection channel is 400 μm.

The inlet reservoir may have a volume of approximately 0.01 mL and the raw sperm sample has a concentration of approximately 100 million sperm per millilitre.

The one or more selection channels may be a single selection channel having a width which is significantly larger than a height of the channel; and
wherein the single selection channel may further comprise a plurality of support structure spaced along a length of the single selection channel to support and prevent a collapsing of the single selection channel.

The microfluidic device may further comprise a base substrate, one side of the base substrate being covered with a layer of a positive photoresist; and the one or more selection channels embedded within the base substrate.

The present disclosure provides a method for preparing the microfluidic device described above, the method comprising the steps of:
printing a pattern of the one or more selection channels onto a photomask using a mask writer;
covering the base substrate with the layer of positive photoresist, said layer having a thickness which is approximately equivalent to the channel dimension;
transferring the pattern of the one or more selection channels from the photomask to the photoresist using a lithographic procedure; and
etching the pattern of the one or more selection channels into the base substrate.

The base substrate may be composed of a rigid, biocompatible material including silicon wafer, rigid glass, polysilicon or nickel.

The positive photoresist may be S1818 or S1822.

The photomask may be a chrome photomask.

The lithographic procedure may include but are not limited to standard photolithography and electron-beam (e-beam) lithography.

The present disclosure provides a method for using the microfluidic device described above, the method comprising the steps of:
filling the microfluidic device with a fluid media such that the selection channels are suitably filled with said fluid media;
loading a semen sample into the inlet reservoir;
loading a fluid media sample into the outlet reservoir;
injecting said semen sample in the inlet at a sample flow rate and, concurrently injecting said fluid sample in the outlet reservoir into the selection channels at a flow rate which is of equal magnitude to the sample flow rate; and
waiting for a time period in a range from 5 to 60 minutes for a slither swimming subset of sperm in said semen sample to traverse through the selection channels from said inlet reservoir to said outlet reservoir.

In an embodiment of the method the fluid media sample is a buffering solution of HEPES, MOPS, TES or Tris, or a solution of human tubal fluid.

A further understanding of the functional and advantageous aspects of the present disclosure can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1A is a schematic showing a typical 3D locomotion mechanism of a sperm where the flagella moves in a helical pattern;

FIG. 1B is a schematic showing a typical 2D, slither-swimming motion of a sperm where the flagella moves in a plane parallel to a surface;

FIG. 2A is a diagram of an embodiment of the microfluidic device;

FIG. 2B is a diagram of a blown-out view of an embodiment of the microfluidic device showing the individual selection channels;

FIG. 3 is a diagram showing the patterns of locomotion for sperm in various locations of an embodiment of the microfluidic device;

FIG. 4A is a superposition of fluorescence images of sperm with channel outlines indicated with dashed lines, and indicating paths of travel for sperm is a superimposed, contrast image of the microfluidic device showing paths of travel for slither swimming sperm;

FIG. 4B is a superposition of brightfield images of sperm, indicating paths of travel for sperm as they transit selection channels;

FIG. 4C is a superposition of brightfield images of sperm, indicating a path of travel for sperm as the sperm transits a selection channel, and with sperm path of travel indicated with an adjacent dashed line;

FIG. 5A is a graphical representation comparing the head DNA % for infertile patient samples;

FIG. 5B is a graphical representation comparing the olive tail moments for infertile patient samples;

FIG. 5C is a graphical representation comparing tail moments for infertile patient samples;

FIG. 5D is a graphical representation comparing the frequency of various head DNA % bins for infertile patient samples;

FIG. 6A is a graphical representation comparing the head DNA % for donor samples;

FIG. 6B is a graphical representation comparing the olive tail moments for donor samples;

FIG. 6C is a graphical representation comparing tail moments for donor samples;

FIG. 6D is a graphical representation comparing the frequency of various head DNA % bins for donor samples;

FIG. 7A is a graphical representation comparing the VSL, VCP and VAP speeds of sperm in the inlet reservoir and selection channels;

FIG. 7B is a graphical representation comparing the LIN and WOB of sperm in the inlet reservoir and selection channels; and

FIG. 7C is a graphical representation comparing the ALH of sperm in the inlet reservoir and selection channels.

### DETAILED DESCRIPTION

Various embodiments and aspects of the disclosure will be described with reference to details discussed below. The following description and drawings are illustrative of the disclosure and are not to be construed as limiting the disclosure. The drawings are not necessarily to scale. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

As used herein, the terms “slithering” and “slither swimming”, refer to a manner of sperm locomotion characterized by a two-dimensional movement of the sperm tail or sperm flagellum. When a sperm is said to be “slithering”, the entire length of the sperm lies within approximately 1 μm of a given surface or substrate, and the sperm tail beats on a plane parallel to the surface. Human sperm tend to swim faster when “slither swimming” and this swimming mode is also characterized by a reduced drag force and increased efficiency of sperm locomotion.

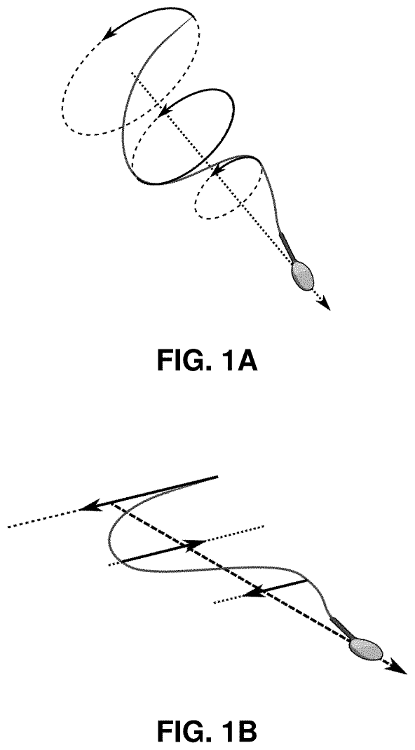

Sperm is a ‘pusher microorganism’ that pushes the fluid away at the back, drags fluid in at the head, and pulls in the fluid from the sides. Interactions of the hydrodynamic flow field of sperm “pushing” with surfaces or the flow field from other sperm influence sperm motion. Patterns of sperm accumulation and locomotion that result from the hydrodynamic interactions of the sperm near surfaces is inherent to natural conception. At in vivo-relevant viscosities, human sperm are known to exhibit multiple patterns of locomotion as shown in FIG. 1A and FIG. 1B. Referring to FIG. 1B, a 2D sperm swimming mode is shown. This pattern of slither swimming occurs when the sperm is almost in contact with a boundary/surface and the helical locomotion of the sperm is restricted. In the apparatus and method proposed herein, the specific slither swimming of the sperm occurs such that the entire length of the sperm body is confined within 1 μm of the surface. In this slither swimming mode, the full sperm body, having an average length in a range from 50-80 μm, is aligned parallel to the boundary/surface such that the sperm's tail or flagellum moves in a 2D plane.

This pattern of locomotion is discernible from previously established patterns of locomotion observed in bulk and near-wall swimming sperm, such as the motion shown in FIG. 1A where the flagellum moves in a 3D rolling helical wave. The slither-swimming of sperm, which is either slithering or slither swimming, generally occurs or is most prevalent in fluids of relatively higher viscosities. In fluids with viscosities that meet or exceed 40 mPa·s, over 65% of human sperm have been shown to exhibit slither mode swimming. In practice, sperm tend to accumulate and navigate near surfaces. Near-surface navigation in vitro has been shown to help sperm swim against a flow.

Sperm are also disposed to swim more rapidly during “slither swimming”, partially as a result of the reduced drag force and increased efficiency of sperm locomotion. Human sperm swim 50% faster in the slither mode than in 3D bulk swimming modes, suggesting a strategy that is well-suited to the highly viscous and confined regions of the reproductive tract. Given the importance of the particular mode of sperm locomotion in natural reproduction, the high viscosity environment of the female reproductive tract, and the prevalence of slither swimming in such conditions, it was hypothesized that slither swimming ability could be linked to overall sperm quality. Specifically, it was hypothesized that slither swimming ability could be indicative of high DNA integrity, where high DNA integrity can be characterized by sperm having a percent of head DNA which is greater than 90% (>90%). The microfluidic device disclosed herein has been constructed to address the challenge of achieving a sperm population for egg fertilization with enhanced DNA integrity by accurately impeding the helical locomotion of sperm and requiring slither-swimming to select a subset of sperm.

The structure of the microfluidic device will first be described in a general form and with particular reference to the movement patterns of individual sperm that the structure of the device requires. The proposed device requires selected sperm to swim using a slither swimming mode of locomotion by impeding the helical locomotion of a subpopulation of sperm. Sperm must slither through an array of microchannels (herein referred to as “selection channels”) to reach the outlet reservoir. Once the sperm in the sample are located within the selection channels, the geometrical confinement of the selection channel (described in detail in a later section) mandates the slither swimming of sperm to pass through the channel.

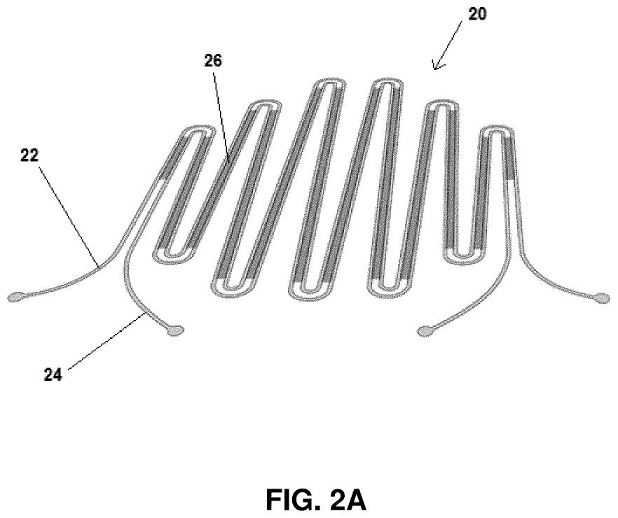

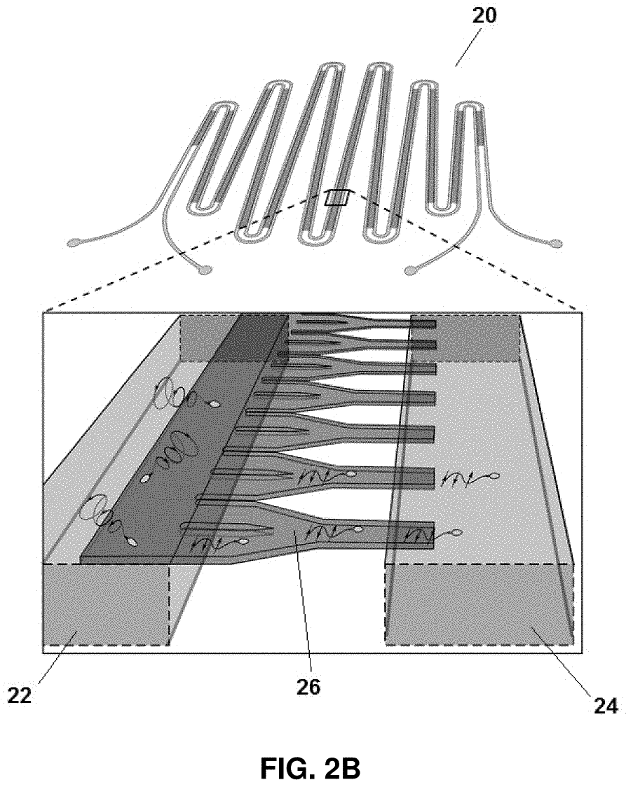

FIG. 2A shows a general embodiment of proposed microfluidic device 20. In the general structure of the device 20 the channel structure comprises a pair of proximally placed inlet and outlet reservoirs 22, 24, which are connected by a series of selection channels 26. The overall form of the inlet and outlet reservoirs may be in a variety of configuration including straight reservoirs, angled reservoirs, curved reservoirs, zig-zag reservoirs or serpentine reservoirs.

Referring to the superimposed view of the device channels as shown in FIG. 2B, the device is composed of an inlet reservoir 22 connected to selection channels. The device also comprises an outlet reservoir 24 connected to selection channels. The inlet reservoir and outlet reservoirs 22, 24 are bridged by a series of selection channels 26 that limit the passage of sperm between the inlet and outlet reservoirs 22, 24. The selection channels 26 are configured to provide a geometric confinement on the sperm where in an embodiment, the channels have a dimension which is slightly longer than the smallest cross-sectional dimension of a typical human sperm head, which has an average diameter of 1.1 μm.

The similarity of the sperm head diameter and channel dimension (with the channel diameter still being slightly larger than the sperm head size) and the long and rectangular construction of the channels will impede helical locomotion of the sperm and force a slither capable subpopulation of the sperm to swim using a slither swimming mode. By requiring the subpopulation of sperm to traverse the channels using a slither swimming mode, the selection channels will only select the slither capable-subset of the sperm within the semen sample. The dimension of the channel such that the selection channel is slightly longer than the average diameter of a sperm head may be in a range from 2-μm to 10-μm

In an embodiment, the selection channel dimension which is similar to the sperm head diameter has a length of approximately 2-μm, where this 2-μm dimension is a height of the channel.

In an additional embodiment, the selection channel dimension is 8 μm. This 8 μm selection channel height does not fully restrict the helical locomotion of the sperm within the selection channels, but provides a significant advantage to the slither capable subset of the sperm population, thereby selecting for slither capable sperm.

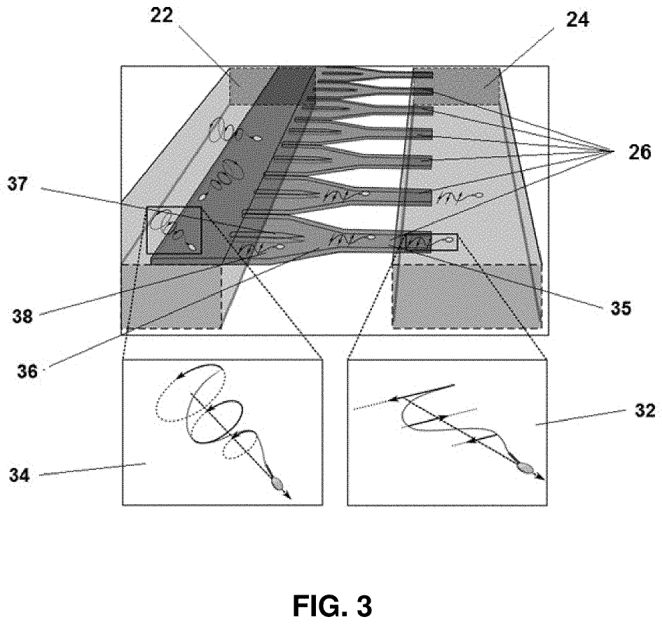

Referring to the device in FIG. 3, the sperm which are capable of moving in the slither-swimming motion will exhibit a motion (within the selection channels) where the entire length of the sperm lies within 1 μm of the surface and the flagella beats on a 2D plane 32 as the sperm moves along the selection channel and into the outlet reservoir. As shown in this representative drawing, the inlet reservoir will contain a semen sample where the sperm are free to swim in the typical, helical manner 34. The selection channels may be configured in variety of orientations/numbers/shapes which will still lead to a functional device.

FIG. 3 provides a specific embodiment of the selection channels with a rectangular, narrowing cross-section where the channel comprises a wider first section 38, tapering second section 36 and a narrow third section 35. In this specific embodiment, the wider, first section 38 of the selection channel are split by a separation vane 37 which guides the movement of the slithering sperm.

In an additional embodiment, the selection channels are rectangular and significantly long relative to the channel height to encourage motile sperm within the sperm sample to access and enter a slither selection channel, if able. The geometry of each selection channel is configured so as to generally guide the sperm across the length of the selection channel from inlet to outlet. The selection channels may also be configured to have an elliptical cross-section such that the channels have at least one dimension of approximately 2 μm.

The selection channels are typically configured to be formed in a planar configuration and to be arranged side-by-side. Each of the rectangular selection channels are preferably 400 μm long and act to guide sperm swimming along the walls. In additional embodiments, the length of the selection channels may be reduced down to approximately 1 μm or lengthened up to approximately 10 cm such that the selection channels would provide a more or less stringent test for selection of slither-capable sperm. Corners in this selection channels may be rounded so that sperm are able to continuously follow the wall across the length of the selection channel.

In a general embodiment, a relatively high concentration of sperm in the inlet reservoir is utilized. The proposed device is capable of screening out up to 99.9% of sperm contained within a sample, at a ratio of 1/1000 sperm passing through the selection channels. For example, a suitable inlet reservoir volume of approximately 0.01 mL and a sperm sample concentration of approximately 100 million sperm per millilitre may achieve a clinically relevant level of sample throughput and sample selection. For a typical experiment with this exemplary concentration within the device, approximately 1 million sperm will initially occupy the inlet reservoir and approximately 1,000 slither-capable sperm will be collected at the outlet reservoir once the selection procedure has been completed.

In an additional embodiment of the proposed device, the device contains exactly 3,250 rectangular selection channels, each with a corresponding inlet connecting the channel to the inlet reservoir and an outlet connecting the channel to the outlet reservoir. In the same embodiment the inlet reservoir is a rectangular chamber with a length of 33 cm, which represents 1,100 cross-sectional channel widths, effectively spreading the sample over 3,250 rectangular selection channel inlets. It will be appreciated that the number of selection channels in the microfluidic device may vary. While it has been found that microchannel arrays with about 200 to about 3,000 selection channel paths generally provide acceptable selection performance, depending on the expected sample volume and the desired approximate number of sperm to be collected, supplementary or fewer selection channels may be provided in variant embodiments.

In an specific embodiment of the embodiment shown in FIG. 3, the selection channels are one single selection channel having a width which is significantly larger that the channel height, and which provides the fluid connection between the inlet reservoir and outlet reservoir. Given the low height-to-width aspect ratio of this embodiment, the single selection channel further includes a plurality of pillars or alternative structure spaced along the length of the channel to support and prevent collapsing of the channel.

In a further embodiment, the silicon wafer is a quarter wafer (a quarter slice of a circular silicon wafer as known in the art) having a width of approximately 33 cm. In this embodiment, the selection channels are a microchannel array of approximately 33,000 selection channels. In this embodiment, the selection channels have a width of approximately 10 μm, (wide enough for sperm to squeeze through). In an additional embodiment, the device contains a microchannel array of a plurality of rows and columns of “stacked” selection channels. This microchannel array may comprise up to 1 million selection channels.

Regarding the makeup and fabrication process for the proposed microfluidic device, a variety of rigid, biocompatible materials are suitable for use as the base constituent during the device's construction. Substrates made up of these various biocompatible materials are utilized to form a rigid base upon which the selection reservoirs and channels can be constructed.

In a first step of the fabrication process, a pattern of the rectangular selection channels is printed on a chrome photomask using a mask writer, such as a UV laser writer or e-beam mask writer. Silicon wafer substrates of approximately 1 mm thickness are then covered with an amount of positive photoresist, such as S1818 or S1822, which is of approximately equivalent thickness to the desired depth/width of the selection channels. The pattern of the rectangular selection channels is then transferred from the photomask to the photoresist by a lithographic procedure, where examples of such lithographic procedures include, but are not limited to standard photolithography and electron-beam (e-beam) lithography. The rectangular selection channels are then etched into the silicon wafer substrate to a suitable depth of between 1-10 μm.

In an embodiment, rigid glass and silicon substrates are used as the base upon which the sperm selection reservoirs and channels can be fabricated. It will be appreciated that the silicon wafers which contain the etched selection channels may have a variety of thicknesses which are greater than 1 mm. It will also be appreciated that the silicon wafer substrate need not be limited to being formed from silicon. Other suitable materials for the formation of this substrate using a similar DRIE etching technique include polysilicon and nickel.

In an additional embodiment, the substrate initially has an approximately flat surface where the selection channels will be formed. In the embodiment, area of the substrate which would surround the selection channels are “grown” into place, as opposed to etching the selection channels into the substrate. The growing of the areas of the substrate surrounding the selection channels could be completed using a chemical vapor deposition (CVD) or physical vapor deposition (PVD) process.

In an embodiment, the process of spin-coating, photolithography and etching processes to form the etched selection channels on the substrate is repeated on the same silicon wafer to fabricate at least a second layer containing the inlet and outlet reservoirs, where each reservoir has a depth of approximately 100 μm. Inlet and outlet ports are then drilled on the wafer. The silicon wafer containing the reservoirs and channels is then anodically bonded to the borosilicate glass plate using a wafer bonder. The etching of the silicon wafer may be completed by a variety of highly anisotropic etching processes which are suitable to create deep penetration and trenches in the chosen wafer and substrate materials. The etching of the selection channels into the substrate may be completed by a variety of etching methods including reactive-ion etching (RIE) or wet etching.

### Device Preparation and Use

In an embodiment of the proposed microfluidic device, the device (including channels and reservoirs) is submerged in a suitable experimental fluid media and vacuumed for a suitable period of time such that the channels are filled with the buffer solution. A variety of suitable fluid media may be used in this step and would be well understood by one skilled in the art. Suitable media include, but are not limited to buffering solutions such as: HEPES, MOPS, TES and Tris, or a solution of human tubal fluid. For the embodiments where the fluid media is a buffering solution, a suitable amount of poly vinyl alcohol and methyl cellulose may be added to the buffer to form a non-Newtonian buffer solution. An exemplary fluid media is a HEPES-buffered solution chemically composed of 135 mM NaCl, 5 mM KCl, 0.6 mM Na2HPO4·2H2O, 25 mM HEPES and 12 mM D-glucose) which is supplemented with 1 mg ml-1 poly vinyl alcohol (to prevent sperm from attaching to surfaces) and 0.5% methyl cellulose.

Once the microfluidic channels are sufficiently filled with buffer, a sealable valve or the like may be temporarily connected to the inlet of each selection channel. The sealable valve may then be used to load a raw semen sample and more experimental buffer. The semen sample and experimental buffer may be loaded into separate glass syringes and placed in fluid connection to an inlet and outlet of each channel of device, respectively. Alternatively these syringes may be plastic syringes or metal syringes.

In this embodiment, the loaded syringes act as external reservoirs connected to the inlet and outlet reservoirs of the microfluidic device. Once the syringes or other form or reservoir are connected to the device, the raw sample and buffer are injected into the device using one or more suitable pumps with suitable flow rates. For example, the semen sample and buffer agent may be injected into the device with two syringe pumps set at 2.0 μl min-1 and 2.2 μl min flowrates, respectively. Once the buffer agent in the inlet of the selection channels are replaced with the raw semen, the flowrate of the pumps is then incrementally reduced to zero to create a no-flow environment.

Once the flow of the fluid media is stopped, a slither-capable subset of sperm will then swim through the selection channels without any induced flow in the fluid media that is contained within the selection channels. In an embodiment, the flow of the fluid media is stopped for a period of 5 to 60 minutes, preferably in a range from 20-30 minutes, allowing time for suitable sperm to swim through the selection channels. It will be appreciated that the length of time that each sperm sample should be allowed to swim through the selection channels without flow in the media will vary depending on the volume and concentration of the sperm sample, and the desired number of sperm in the sample contained in the outlet reservoir. Once the sperm samples are provided time to travel through the selection channels, the sperm sample may then be collected by injecting raw semen and experimental buffer in the inlet and outlet reservoirs, respectively, with similar flowrates used in the loading process as described above, so as to prevent the occurrence of crossflow from the inlet to the outlet reservoir during the collection of the sample. The device is preferably maintained at a constant temperature throughout the duration of the loading, sample swimming and sample collection process. For example, the device may be kept at 37° C. throughout the experiment using a warm plate.

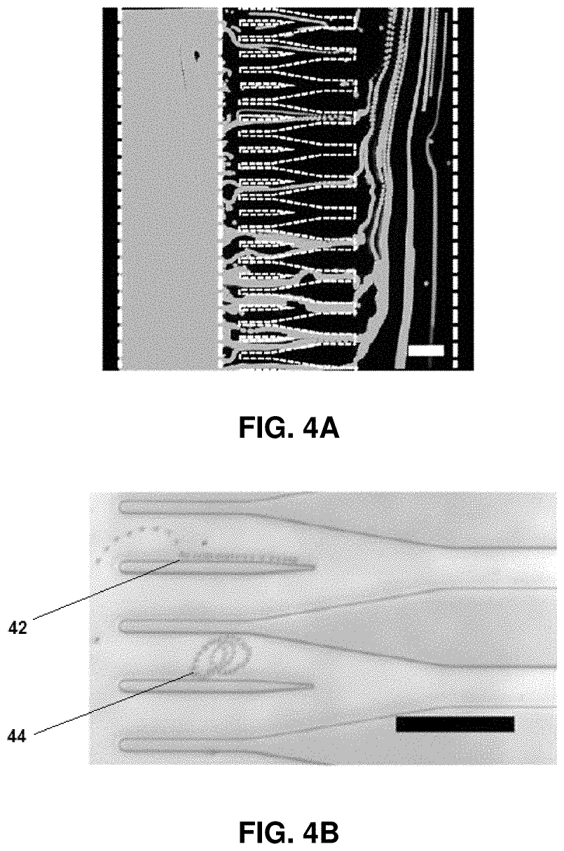

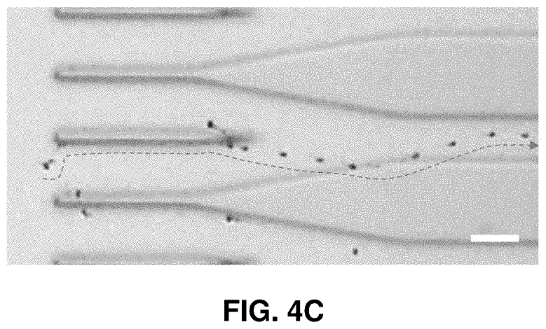

Referring to FIG. 4A, FIG. 4B and FIG. 4C the superimposition of images taken of an embodiment of the device demonstrates the movement of sperm within a sample during a typical selection process. FIG. 4A illustrates the various path taken by sperm which are capable of slither swimming and travel from left-to-right and move from the inlet reservoir, through the entrance to the selection channel inlets and through the narrowing sections of the selection channels to the outlet reservoir. A limited number of sperm from the sample are able to enter and traverse a selection channel by slither swimming. Sperm typically travel within the selection channels using the patterns as shown in FIG. 4B by slither-swimming in either the continuous boundary-following paths along the selection channel walls 42 or by using more circular trajectories 44. It will be appreciated that these two methods of transit are merely examples of successful mechanisms of sperm locomotion, and are by no means the only path that slithering sperm may traverse. FIG. 4C illustrates the path taken by a sperm, where a continuous boundary-following path along the selection walls is used for part of the trajectory.

The sperm contained within the selection channels and allowed to swim across the channels will generally travel at similar speeds throughout the length of the swimming in the stationary media. To more accurately quantify the motion of the sperm in the sample, the motion of each sperm was monitored and measured. Images of the sperm moving through channels were processed to quantify the swimming parameters of sperm inside and outside the selection channels.

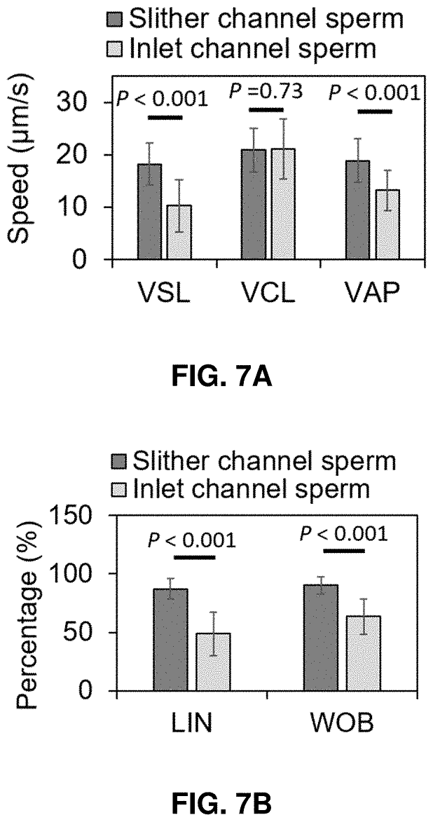

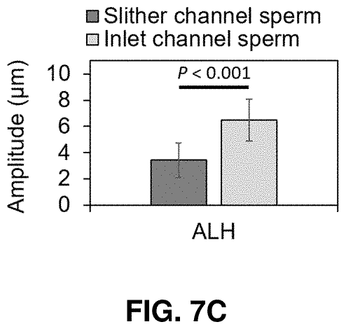

The selection channel boundaries typically guide sperm in a straighter path in comparison to bulk or near wall swimming. In a non-limiting, exemplary study which tracked the motion of inlet reservoir and selection channel sperm, sperm within the selection channel demonstrated increased straight line (VSL) and average path (VAP) velocities as shown in FIG. 7A. Sperm swimming within a selection channel will generally be slithering, whereas sperm swimming along the inlet reservoir may be characterized by either slithering or near-wall swimming. Moreover, sperm generally employ straighter paths in the slither selection channels as measured by linearity (LIN) and wobble (WOB) as shown in FIG. 7B. Also, amplitude measurements (ALH) of sperm locomotion shown in FIG. 7C indicate that the lateral head displacement (ALH) of sperm in a selection channel deviate less from the average path than sperm in the inlet reservoir. In the exemplary analysis of sperm locomotion shown in FIG. 7A-7C, the locations of sperm head centroids were used to calculate straight line velocity (VSL), defined as the distance between the first and the last point tracked divided by the total duration of tracking. Curvilinear velocity (VCL) is the sum of distances between two consecutive sperm positions divided by the corresponding time difference. Average path velocity (VAP) is the average velocity of sperm along the average path. Linearity (LIN) is defined as VSL/VCL.

Exemplary studies which incorporated the proposed device are disclosed herein, and involved the isolation of a subpopulation of human sperm that were able to enter and navigate device channels which prohibit the rotation of the sperm head and necessitate slither swimming.

A series of experiments were completed to validate the ability of the proposed device to select a 2D, slither-swimming capable subpopulation of sperm. To isolate the effect of slither-swimming from the general swimming ability of a sperm cell, an otherwise identical microfluidic platform to the proposed device, with a selection channel height of 30 μm, was constructed, and primed in an identical manner to the microfluidic device having the 2 μm selection channel height. In this device, sperm were not confined to a slither swimming mode and were free to swim the equivalent distance in three-dimensional space (3D selection). The capability of improving sperm DNA integrity by selecting based on slither-swimming ability, which is achieved by the proposed device, can be further validated by comparing the DNA integrity of the device sample to the DNA integrity of sperm samples acquired via traditional, clinical methods such as density gradient centrifugation used in conjunction with a swim-up assay.

Based on numerous, exemplary experiments on the device disclosed herein, it is shown that the slither-capable subpopulation of sperm possesses DNA integrity superior to the motile or general sperm populations, both in high-quality (donor) and low-quality (infertile) semen samples. By confining sperm motion to a 2D plane, the device selects for a slither-capable subpopulation of sperm that possess enhanced DNA integrity over both the motile and general sperm populations. Slithering represents a viable selection mechanism, readily applicable to clinical workflows with the potential to improve outcomes for couples and offspring.

### EXAMPLES

The invention can be further understood by the skilled person with reference to the following examples, which are exemplary, and the inventors' technology is not limited in scope by the exemplified embodiments. Various modifications of the present technology in addition to those described herein will become apparent to those skilled in the art from this description and accompanying figures.

### Example 1

### DNA Integrity Testing Using Comet Assay

The DNA integrity of sperm samples was tested using the comet assay as previously described. In short, 10 μl of selected sperm acquired through each treatment was resuspended in 100 μl of 1% low melting temperature agarose solution at 37° C. The cell suspensions were deposited in 10 μl droplets on a GelBond Agarose gel support film and placed at 4° C. for 10 min for gelation. The film was submerged in the lysis buffer (2.5 M NaCl, 100 mM EDTA, 10 mM Tris, 10% DMSO, 1% Triton X 100, 0.1% DTT) overnight at 4° C. The lysis buffer was replaced with Proteinase K solution (2.5 M NaCl, 100 mM EDTA, 10 mM Tris, 0.1% DTT, 5 mg Proteinase K) and incubated for 30 min at 37° C. The film was rinsed and cooled to room temperature and was then submerged in Alkali solution at 12.4 pH for 45 min. The film was then transferred to a dish with 1×TBE buffer for 10 min and was then electrophoresed at 18 V for 30 min in a horizontal tank. The sample was then stained with SYBR. The DNA integrity of the samples from the proposed device were benchmarked against tests using best clinical practices, where density gradient centrifugation is typically used in conjunction with the swim-up assay (DG+SU). Semen samples were purified through two-layer density centrifugation (300×g for 20 min) of PureSperm®40 and PureSperm®80. The supernatant was discarded, and the sperm pellet was resuspended and centrifuged again (500×g for 10 min) in 3 ml of PureSperm®Wash. Sperm were resuspended and laid below 3 ml of PureSperm®Wash and placed at a 45° angle in an incubator operating at 37° C. with 100% humidity and 5% CO2 for one hour. A 10 μl cell suspension at the top of the solution was extracted for DNA integrity testing.

### Example 2

### Studies on Infertile Patient Sample DNA Integrity

The DNA integrity of infertile patients or those who have difficulty conceiving is considerably lower than a typical donor set from the general population. The proposed microfluidic device is capable of improving the DNA integrity of sperm for infertile patients. Improvement from the original 18% chance (raw infertile sample) to a 77% chance (for slithering sperm) that a sperm picked randomly from the sample possesses high (>90%) head DNA.

Again, the DNA integrity of a usable, infertile sample from the proposed device (which selects for 2D slithering) was compared against the DNA of a usable, infertile sample for the 3D swimming embodiment of the device described previously. The sperm sample selected using the proposed “slithering” device demonstrated higher levels and improved frequency of high DNA over the 3D swimming embodiment, again demonstrating the benefits of slither-swimming for selecting high DNA integrity sperm.

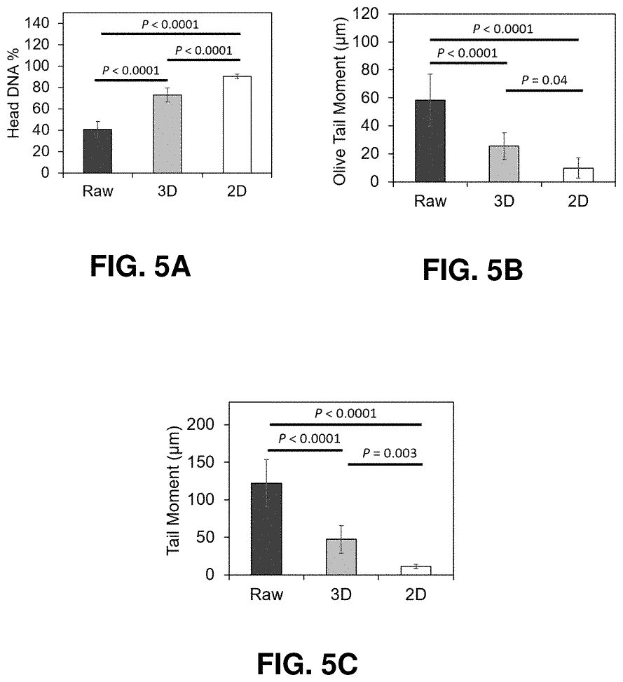

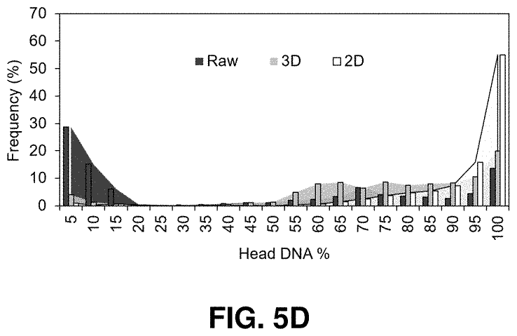

Referring to FIG. 5A-5D these figures provide a comparison of the DNA integrity of sperm selected via 2D slithering to the raw semen as well as a 3D-selected subpopulation of patient samples. The 2D-selected slithering subpopulation exhibits significantly higher head DNA % as well as olive tail moment and tail moment compared to the 3D and raw populations. The average head DNA % distribution of patient samples is shown in FIG. 5D. From an original raw population with only 18% of sperm with high (>90%) head DNA content, 77% of the 2D-selected slithering sperm exhibit high (>90%) head DNA.

### Example 3

### Studies on Donor Sample DNA Integrity

The proposed device was also utilized in an exemplary study on fertile, donor sperm. For each healthy donor semen sample, the DNA integrity of the raw semen, a subpopulation selected via slither swimming (2D), a motile subpopulation (3D), and a subpopulation selected through DG+SU were tested using single-cell gel electrophoresis, a technique well known in the art. DNA integrity was chosen as the sperm quality measure as it is quantitative and is associated with fertilization rate, embryo development, miscarriage, and live birth rate.

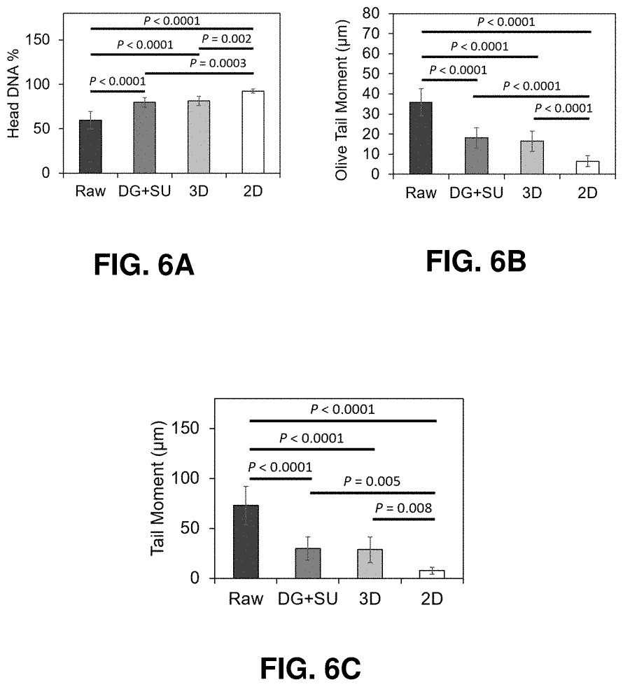

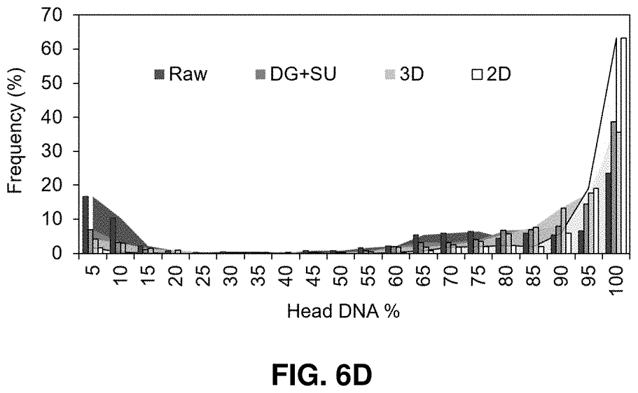

Referring to FIG. 6A-6D, the head DNA %, a measure of DNA integrity, as well as the olive tail moment and tail moment, two measures of DNA fragmentation, for the four aforementioned cases are provided. All three measures indicate a significantly higher DNA integrity of 2D slithering sperm compared to that of 3D motile (P<0.01), raw (P<0.001), and density gradient and swim-up (P<0.01) selected subpopulations. FIG. 6D shows the average distribution of the head DNA %. In the case of 2D selected sperm, 68% of the sperm showed >90% head DNA, whereas in 3D selection only 48% of sperm showed >90% head DNA, and in DG+SU only 45% of sperm showed >90% head DNA.

To summarize, these exemplary studies demonstrate that in healthy donor and infertile-donor patients, a 2D-selection of slithering sperm samples shows improved DNA integrity over motile and raw infertile populations. Thus, the introduction of the proposed microfluidic device, which selects based on the 2D slither swimming ability of a sperm sample and impedes helical locomotion of the subpopulation of sperm, will be beneficial for improving the quality of sperm samples for both healthy and infertile populations. This improvement is highly beneficial from a clinical perspective, where selection of a single good sperm is critical.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

Therefore what is claimed is:

1. A microfluidic device for isolating sperm of a desired quality, the device comprising:
a) an inlet reservoir which for holding a raw sperm sample;
b) an outlet reservoir for collecting sperm separated from the sample; and
c) one or more selection channels disposed between said inlet reservoir and said selection channels to provide fluid communication between the inlet reservoir and outlet reservoir, the one or more selection channels being geometrically configured to impede helical locomotion of a subpopulation of sperm and allow 2D slither swimming of a slither capable subset of sperm within the said one or more selection channels.

2. The microfluidic device of claim 1 wherein the one or more selection channels have a dimension that is sufficiently low so as to restrict the helical locomotion of sperm within said one or more selection channels.

3. The microfluidic device of claim 2 wherein the one or more selection channels have a channel dimension in a range from 1.0 μm to 10.0 μm.

4. The microfluidic device of claim 1 wherein a length of each of said one or more selection channels is in a range from 1 μm to 10 cm and encourages a slither swimming subset of sperm to traverse said one or more selection channels.

5. The microfluidic device of claim 4 wherein the one or more selection channels have a channel dimension which is a channel height of 2 μm, and wherein the length of each selection channel is 400 μm.

6. The microfluidic device of claim 1 wherein said inlet reservoir has a volume of approximately 0.01 mL and said raw sperm sample has a concentration of approximately 100 million sperm per millilitre.

7. The microfluidic device of claim 1 wherein said one or more selection channels is a single selection channel having a width which is significantly larger than a height of the channel; and
wherein the single selection channel further comprises a plurality of support structure spaced along a length of said single selection channel to support and prevent a collapsing of said single selection channel.

8. The microfluidic device of claim 3 wherein the microfluidic device comprises a base substrate, one side of said base substrate being covered with a layer of a positive photoresist; and the one or more selection channels embedded within said base substrate.

9. A method for preparing the microfluidic device of claim 8, the method comprising the steps of:
printing a pattern of the one or more selection channels onto a photomask using a mask writer;
covering the base substrate with the layer of positive photoresist, said layer having a thickness which is approximately equivalent to the channel dimension;
transferring the pattern of the one or more selection channels from the photomask to the photoresist using a lithographic procedure; and
etching the pattern of the one or more selection channels into the base substrate.

10. The microfluidic device of claim 9 wherein the base substrate is composed of a rigid, biocompatible material including silicon wafer, rigid glass, polysilicon or nickel.

11. The microfluidic device of claim 9 wherein the positive photoresist is S1818 or S1822.

12. The microfluidic device of claim 9 wherein the photomask is a chrome photomask.

13. The microfluidic device of claim 9 wherein the lithographic procedure include but are not limited to standard photolithography and electron-beam (e-beam) lithography.

14. A method for using the microfluidic device of claim 1, the method comprising the steps of:
filling the microfluidic device with a fluid media such that the selection channels are suitably filled with said fluid media;
loading a semen sample into the inlet reservoir;
loading a fluid media sample into the outlet reservoir;
injecting said semen sample in the inlet at a sample flow rate and, concurrently injecting said fluid sample in the outlet reservoir into the selection channels at a flow rate which is of equal magnitude to the sample flow rate; and
waiting for a time period in a range from 5 to 60 minutes for a slither swimming subset of sperm in said semen sample to traverse through the selection channels from said inlet reservoir to said outlet reservoir.

15. The method of claim 14 wherein the fluid media sample is a buffering solution of HEPES, MOPS, TES or Tris, or a solution of human tubal fluid.
